(19) United States
(12) Patent Application Publication
(10) Pub. No.: US 2023/0201190 A1
(43) Pub. Date: Jun. 29, 2023

(54) USE OF MULTIFUNCTIONAL LIGANDS FOR TREATING THE RESPIRATORY DISTRESS AND CYTOKINE STORM SYNDROMES ASSOCIATED WITH CORONAVIRUS VIRAL INFECTIONS

(71) Applicants: Gaëtan TERRASSE, MONTCEAU-LES-MINES (FR); H4 ORPHAN PHARMA, DIJON (FR)

(72) Inventors: Gaëtan TERRASSE, MONTCEAU-LES-MINES (FR); Catherine BUR, ORGERUS (FR)

(73) Assignees: Gaëtan TERRASSE, MONTCEAU-LES-MINES (FR); H4 ORPHAN PHARMA

(21) Appl. No.: 17/927,442

(22) PCT Filed: Jun. 4, 2021
(86) PCT No.: PCT/EP2021/065045

§ 371 Date: Nov. 23, 2022

(30) Foreign Application Priority Data
Jun. 4, 2020 (FR) 2005858

(51) Int. Cl. A61K 31/4741; A61P 37/02; A61P 31/14

(52) U.S. Cl. CPC A61K 31/4741; A61P 37/02; A61P 31/14

(57) ABSTRACT

The enantiomers of AMINO-7 TRIETHOXY-4,5,6 OXO-1 DIHYDRO-1,3 ISOBENZOFURANNYL-3)-1 METHOXY-8 METHYL-2 METHYLENEDIOXY-6,7 TETRAHYDRO-, 2,3,4 ISOQUINOLINE or tritoqualine and deuterated derivatives thereof, capable of preventing and treating cytokine storm and respiratory distress in coronavirus infections.

### TECHNICAL FIELD

The present invention relates to the use of multifunctional ligands based on chemical substances, namely the levorotatory and dextrorotatory enantiomers of 1-(7-AMINO-4,5,6-TRIETHOXY-1-OXO-1,3-DIHYDRO-3-ISOBENZOFURANNYL)-8-METHOXY-2-METHYL-6,7-METHYLENEDIOXY-1,2,3,4-TETRAHYDRO-ISOQUINOLIN and deuterated derivatives thereof for the treatment of coronavirus infection-related cytokine storm and pulmonary complications.

### BACKGROUND

Multifunctional ligands are therapeutic molecules with affinity for multiple pharmacological targets at therapeutic doses of 10 nanomolar to one micromole. These pharmacological targets have to be synergistic in order to refer to them as multifunctional ligands. This synergy allows very high activity with little or no side effects. The inventors have demonstrated that 1-(7-AMINO-4,5,6-TRIETHOXY-1-OXO-1,3-DIHYDRO-3-ISOBENZOFURANNYL)-8-METHOXY-2-METHYL-6,7-METHYLENEDIOXY-1,2,3,4-TETRAHYDRO-ISOQUINOLIN is a new class of multifunctional ligands.

The novel 2019 coronavirus (COVID-19) pneumonia initially broke out in Wuhan, China, in early December 2019 and spread nationwide within two months. Within China, the COVID-19 outbreak is subsiding, but outside of China, it is gaining exponential momentum. European countries (Italy, Spain, Germany, France), the United States of America, Iran and South Korea face enormous challenges in dealing with COVID-19, which has been officially declared a pandemic by the World Health Organisation on 11 Mar. 2020.

Coronavirus infectious disease 2019 or Covid-19 (originally known as 2019-nCoV) is an emerging infectious zoonotic viral disease caused by the SARS-CoV-2 coronavirus strain.

The most common symptoms thereof are fever, cough and difficult respiration and, more rarely, acute respiratory distress syndrome (ARDS), which can cause death, in particular in people who are frail due to age or comorbidities. Another fatal complication is an exacerbated response of the innate immune system (cytokine storm).

There is a high rate of asymptomatic forms.

Human-to-human transmission is mainly via respiratory droplets, sputum as for seasonal influenza, especially during coughing and sneezing or by hand contact with a contaminated surface followed by touching the face (mouth, nose, eyes, not skin). The incubation period usually lasts two to fourteen days, or even 20 days (five days on average).

A significant proportion of infected people, including children, often show no symptoms but can transmit the disease, increasing its contagiousness. A French study published on 27 Mar. 2020 describes three types of ill people:
Patients with few clinical signs but with a high nasal viral load and being highly contagious;
Patients with mild symptoms at the start but worsening around day ten with the onset of severe acute respiratory syndrome despite a decreasing viral load. The immune reactivity in the lungs is said to be unregulated
Patients with a rapid worsening to acute respiratory syndrome with persistent high viral load in the nose and throat and the appearance of SARS-Cov-2 blood viremia causing multi-visceral failure leading to death. This third type of ill person especially affects the elderly.

Main complications of hospitalised patients are acute respiratory distress in 30% of cases, significant release of cytokines responsible for secondary haemophagocytic lymphohistiocytosis which is a particular form of cytokine storm in 50% of cases.

Bacterial lung superinfection in 10% of cases. Myocarditis in 10% of cases.

It is very important to understand that it is at the moment when the viral load has decreased and becomes lower, that the risk of a cytokine storm becomes the highest.

This proves that the storm is not related to the multiplication of the virus but to a response of the immune system as a result of the infection.

The cytokine storm is similar to anaphylactic shock in allergy: a very low viral load is sufficient to cause a fatal reaction, the main cause of which is local degranulation of basophils in the lungs. Indeed, it has been demonstrated that fatal cases of asthma are due to basophils. (Kepley 2001 Immunohistochemical detection of human basophils in postmortem cases of fatal asthma). On the other hand, double-stranded RNA, equivalent to coronavirus infection, has been shown to activate basophil degranulation (Ramadan 2013 Activation of basophils by the double-stranded RNA poly(A:U) exacerbates allergic inflammation).

While cytokine storm is an important cause of mortality in coronavirus infection, pulmonary complications such as acute or chronic fibrosis should not be overlooked. Many survivors of the 2003 severe acute respiratory syndrome (SARS) outbreak developed residual pulmonary fibrosis with increased severity observed in older patients. Autopsies of patients who died of SARS have also shown varying degrees of fibrosis. Pulmonary fibrosis can sometimes be seen as a consequence of several respiratory viral infections, but it is much more frequent after infection with the SARS coronavirus (SARS-CoV). Thus it appears that coronaviruses are frequently involved in the occurrence of pulmonary fibrosis.

Today there is no treatment for cytokine storm, nor is there any treatment to prevent pulmonary fibrosis.

No currently registered drug has been shown to be effective.

Clinical trials are underway, but no conclusive results are available. Furthermore, there is little chance of a vaccine due to the number of mutations in the virus.

Clinical research on the new coronavirus (COVID-19) continues to intensify in France. On 10 Apr. 2020, the French Drug Safety National Agency (ANSM for “Agence Nationale de Sécurité des Medicaments”) indicated that it had authorised 35 clinical trials in France for the management of patients suffering from COVID-19, 78% of which were conducted by academic sponsors.

In addition, France is involved in 7 of the 10 major international trials currently underway. All the therapeutic or non-therapeutic interventions authorised or under investigation are listed by the Ministry of Health.

The WHO international registry currently has over 1100 clinical trials, of which 654 are interventional trials and 261 are randomised studies. That is to say almost 500 new studies registered in around three weeks. Although research has never been so intense and rapid, solid data is still scarce thereby making therapeutic prospects still unclear. In its recent summary of knowledge, the Operational Coordination of Epidemic and Biological Risk (COREB for “Coordination Opérationnelle Risque Epidémique et Biologique) mentioned the lack of current data regarding treatments expected to be effective in the management of patients with Covid-19 (opinion from French HCSP (“Haut Conseil de la Santé Publique”)), and which should therefore be reserved for evaluation in clinical trials.

Initial data from an open-label randomised clinical trial of Lopinavir®-Ritonavir®, published in the course of March, turned out to be negative. However, other clinical trials—including DISCOVERY in severe patients or COVIDAXIS in prophylaxis for caregivers in France—are ongoing.

The only data relating to REMDESIVIR® are those from a small observational cohort of severe cases admitted to the intensive care unit, which described an improvement in oxygen requirements. These data are questionable given the lack of a control group.

OSELTAMIVIR® has been shown to be ineffective both in vitro and clinically in the management of COVID-19. UMIFENOVIR® (or Arbidol), currently registered in Russia and China for the treatment of influenza, continues to be studied with a stronger rationale (interaction with the virus upon binding to the ACE2 receptor). For the time being, existing data are still based on an observational study whose conclusions must be confirmed by comparative data, the most recent of which (see above) are negative. The antiviral treatment pathway seems unpromising. The vaccine route could lead to a vaccine, but probably with low preventive efficacy. Moreover, it would be necessary to have a vaccine coverage higher than 80% to limit spread of coronavirus. This seems unlikely. The anti-IL6 pathway only covers part of the cytokine storm. For the sequelae of coronavirus, namely induced fibrosis, there are treatments but with many side effects. Two drugs are currently on the market, but for idiopathic pulmonary fibrosis. These are Pirfenidone and Nintedanib.

According to the French High Health Authority HAS (Haute Autorité de Santé), the efficacy of Pirfenidone has been assessed according to an intermediate criterion evaluating lung function and being a marker of disease progression. The difference observed on this criterion is in favour of Pirfenidone compared to placebo, but this difference is small, of unknown clinical significance and heterogeneous from one study to another.

The effect observed with ESBRIET® on the decline in lung function in patients with specific functional endpoints (FVC≥50% and DLco≥35%) is small. The clinical benefit for patients with idiopathic pulmonary fibrosis is difficult to assess, as clinically relevant endpoints (quality of life, overall survival, etc.) have been subject to exploratory and non-robust analyses. However, patient pooling studies have revealed a significant improvement in survival in some patient profiles.

Main adverse effects of Pirfenidone observed were gastrointestinal disorders (nausea, diarrhoea, dyspepsia), skin disorders (photosensitisation and rash) and metabolic and nutritional disorders (anorexia and loss of appetite). In view of the side effects, it seems unlikely that this product will be used for COVID 19-induced fibrosis.

On the other hand, Nintedanib known under the trade name OFEV® has been approved in the USA and Europe for the treatment of idiopathic pulmonary fibrosis. The mode of action of Nintedanib is that of a tyrosine kinase inhibitor. This product is used at a dose of 200 to 400 mg/day. Many, in particular gastrointestinal, side effects have been observed.

According to the HAS, “the medical benefit” by OFEV® is moderate.

Given the efficacy of Nintedanib, assessed according to an intermediate criterion, with a moderate number of effects compared to placebo, and the methodological limitations of the comparative analysis of mortality, OFEV® provides, like ESBRIET®, a minor improvement in medical benefit (ASMR IV “Amelioration du Service Medical Rendu”), in patients with idiopathic pulmonary fibrosis confirmed on clinical, radiological and/or histopathological parameters, whose respiratory functional criteria are as follows: FVCp≥50% and DLco≥30%. Side effects of OFEV® are even more numerous than those of ESBRIEST®. The HAS notes nearly 92% of side effects. For these reasons, the treatment of post COVID 19 fibrosis by these two drugs seems unlikely at the therapeutic doses used today.

A number of products are currently being studied for the treatment of idiopathic pulmonary fibrosis:

PLN-74809, from PLIANT therapeutics, is a dual av86 and av81 integrins-selective inhibitor developed for the treatment of idiopathic pulmonary fibrosis (IPF) and primary sclerosing cholangitis (PSC). PLN-74809 has been shown to inhibit TGF-β activation by up to 70% in alveolar macrophages from healthy volunteers in a dose and exposure dependent manner. It is an oral product. There are no efficacy results in humans for this product. A phase II trial is underway but no results have been published.

BBT-877 from BRIDGEBIO THERAPEUTICS, a potent autotaxin inhibitor in clinical development for the treatment of idiopathic pulmonary fibrosis, has provided pharmacokinetic, pharmacodynamic and safety results from a randomised, double-blind, placebo-controlled Phase I clinical study. Although the product is in phase II, there are no efficacy data for this product. Only preclinical results in animals are available.

Galecto's lead candidate is TD139, a galectin-3 selective inhibitor. When inhaled by IPF patients, TD139 is designed to block galectin-3 and thus prevent the galactoside-binding lectin from triggering a pathway that activates macrophages and myofibroblasts.

MN-001 from MEDICINOVA (Tipelukast®) is a leukotriene (LT) receptor antagonist and phosphodiesterase (PDE) inhibitor (primarily 3 and 4) and 5-lipoxygenase (5-L0) inhibitor. The drug is currently in phase II, but results are reported to be of poor efficacy without improvement in fibrosis. GLPG1690, from GALAPAGOS, is a once-daily oral autotaxin inhibitor currently in phase 3. The results highlight a halt in disease progression but no improvement in pulmonary fibrosis. Novartis' VAY736 is a fully human IgG1 monoclonal anti-B cell activating factor (BAFF) receptor antibody designed for direct ADCC-mediated B-cell depletion, providing a dual mode of action. Currently in phase II. The product was discontinued due to lack of efficacy.

KD025 (also known as SLX-2119) is a kinase 2 inhibitor that is being tested for the treatment of idiopathic pulmonary fibrosis (IPF) by the biopharmaceutical company Kadmon. Currently this product is stopped in phase II clinical trials.

In conclusion, there is currently no product to treat fibrosis despite numerous ongoing clinical trials and even less to prevent the occurrence of fibrosis in patients with coronavirus infection.

Tritoqualine is a chemical substance that has been known for many years and used as an antihistamine. Its manufacture is described in French patent FR 1.295.309.

Tritoqualine is 7-amino-4,5,6-triethoxy-3-(5,6,7,8-tetrahydro-4-methoxy-6-methyl-1,3-dioxolo[4,5-g]isoquinolin-5-yl)phthalide. In its marketed pharmaceutical form, it is in the form of a mixture of enantiomers. Tritoqualine is known for its anti-allergic activity through its inhibitory action on histidine decarboxylase. However, this activity is very weak and does not explain numerous properties it has on various clinical symptoms, rhinitis, urticaria, eczema, mastocytosis.

The activity of tritoqualine has been demonstrated in cystic fibrosis (EP2854947, EP2844253). Although it is referred to as fibrosis, it is in fact a genetic disease, the pathophysiology of which is due to the deficiency of electrolyte transport via an abnormal CFTR. There is no fibrosis as such, but thickening of mucosal secretions due to deficient water transport.

The activity of tritoqualine has also been described in idiopathic pulmonary fibrosis (patent EP3429587).

There is also a description of the action of tritoqualine on liver and collagen secretion (suppressive effect of tritoqualine on cell growth and collagen secretion in fibroblast. Omezu. 1986).

Basophils are the least abundant granulocytes, representing less than 1% of peripheral blood leukocytes. For this reason, as well as their poor phenotypic and morphological characteristics and the lack of animal models, their physiological specificity during the immune response has been ignored until recently. Although both mast cells and basophils are the primary effector cells in bronchial asthma, there is immunological, biochemical and pharmacological evidence that they perform distinct functions at the onset and during the course of asthma (karasumaya 2011). Basophils are indeed the first major producers of IL-4 and IL-13, which are essential for initiating and maintaining an allergic response (Casolaro 1990). Moreover, in line with their putative effector they are clearly associated with fatal asthma (Kepley 2001 and Koshino 1993); basophils are detected in nasal lavage fluid after provocative testing in patients with allergic rhinitis (Iliopoulos 1992). This notion is supported by the correlation between IL-4R gain-of-function mutations and exacerbation of allergic disease.

Recent studies have shown that basophil activation is not only promoted by cross-linking of gene-specific IgEs, but can also be promoted by non-sensitised individuals by parasitic antigens, lectins and superantigenic viruses, either by direct cross-linking of FcεRI or by binding to non-specific IgE antibodies (Falcone 2006).

Viral respiratory infections, in particular those induced by rhinovirus and respiratory syncytial virus, are the most common and important cause of acute asthma exacerbation in adults and children and represent a global health burden (Jackson 2010, Stensballe 2009). A growing body of evidence supports the hypothesis that these infections cause more morbidity in asthmatics than in healthy individuals.

It is probable that coronaviruses have a more toxic effect on respiratory function and that asthma is not the most important factor. An English study demonstrated that age was the main risk factor for death (160 times greater risk of death over 80 years). Understanding the mechanisms responsible for virus-induced airway inflammation will help to find adapted therapeutic responses.

The inventors postulated that coronaviruses stimulate the TLR4, TLR3 and TLR7 pathways to induce cytokine storm via basophils. The activated basophils then stimulate eosinophils and macrophages to amplify the immune response, leading to respiratory distress and death in the virus-infected patient. It is probably the viral proteins that stimulate TLR4. Then the cascade passing through MyD88 will lead to the secretion of the cytokines TH2, IL6, IL4 and IL13.

### SUMMARY

The present invention also relates to 7-amino-4,5,6-triethoxy-3-(5,6,7,8-tetrahydro-4-methoxy-6-methyl-1,3-dioxolo[4,5-g]isoquinolin-5-yl)phthalide for use in the treatment of coronavirus infection-related cytokine storm.

The present invention also relates to 7-Amino-4,5,6-triethoxy-3-(5,6,7,8-tetrahydro-4-methoxy-6-methyl-1,3-dioxolo[4,5-g]isoquinolin-5-yl)phthalide for use in the prevention of coronavirus infection-related cytokine storm.

The present invention also relates to 7-Amino-4,5,6-triethoxy-3-(5,6,7,8-tetrahydro-4-methoxy-6-methyl-1,3-dioxolo[4,5-g]isoquinolin-5-yl)phthalide and deuterated derivatives thereof for use in the treatment of coronavirus infection-related respiratory distress.

The present invention also relates to 7-amino-4,5,6-triethoxy-3-(5,6,7,8-tetrahydro-4-methoxy-6-methyl-1,3-dioxolo[4,5-g]isoquinolin-5-yl)phthalide and deuterated derivatives thereof for use in the prevention of coronavirus infection-related respiratory distress.

According to one preferred embodiment of the invention, 7-Amino-4,5,6-triethoxy-3-(5,6,7,8-tetrahydro-4-methoxy-6-methyl-1,3-dioxolo[4,5-g]isoquinolin-5-yl)phthalide for use in the treatment of cytokine storm is remarkable in that it is used in a dose of 0.1 mg/kg/day to 10 mg/Kg/day.

According to one preferred embodiment of the invention, 7-Amino-4,5,6-triethoxy-3-(5,6,7,8-tetrahydro-4-methoxy-6-methyl-1,3-dioxolo[4,5-g]isoquinolin-5-yl)phthalide for use in the prevention of cytokine storm is remarkable in that it is used at a dose of 0.1 mg/kg/day to 10 mg/Kg/day.

According to one preferred embodiment of the invention, 7-Amino-4,5,6-triethoxy-3-(5,6,7,8-tetrahydro-4-methoxy-6-methyl-1,3-dioxolo[4,5-g]isoquinolin-5-yl)phthalide for use in the treatment of cytokine storm is remarkable in that it is used at a dose of 5 to 700 mg/day.

According to one preferred embodiment of the invention, 7-Amino-4,5,6-triethoxy-3-(5,6,7,8-tetrahydro-4-methoxy-6-methyl-1,3-dioxolo[4,5-g]isoquinolin-5-yl)phthalide for use in the treatment of cytokine storm is remarkable in that it is packaged in the form of soft gelatin capsules, tablets, capsules, syrup or gel.

### BRIEF DESCRIPTION OF THE FIGURES

FIG. 1 illustrates the presence of the asymmetric carbons, which are noted as A and B.

FIG. 2 illustrates the form of the RR isomer.

FIG. 3 illustrates the form of the SS isomer.

FIG. 4 illustrates the methyls that can be deuterated on tritoqualine

FIG. 5 illustrates the variation in resistances in treated and untreated mice

FIG. 6 illustrates the variation in resistances at 2 minutes between the 3 groups of mice

FIG. 7 illustrates the variation in resistances at 6 minutes between the 3 groups of mice

FIG. 8 non-degranulated basophils

FIG. 9 degranulated basophils

FIG. 10 effect of tritoqualine on the basophil degranulation inhibition.

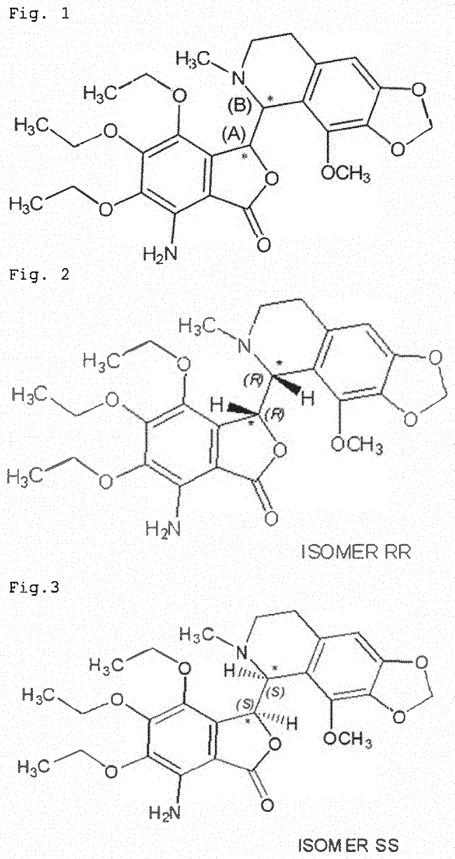

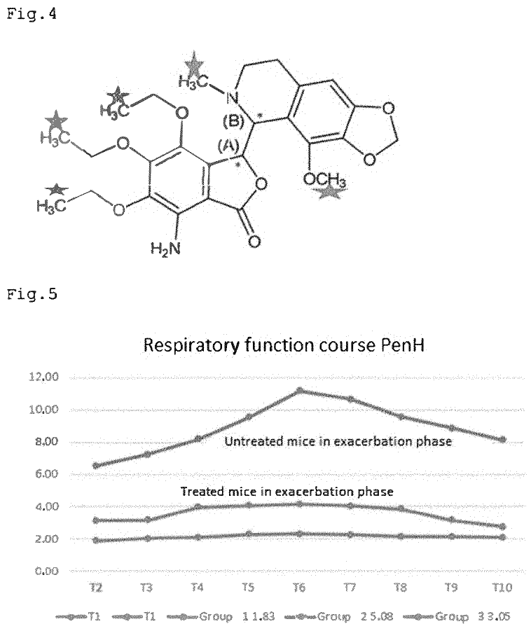

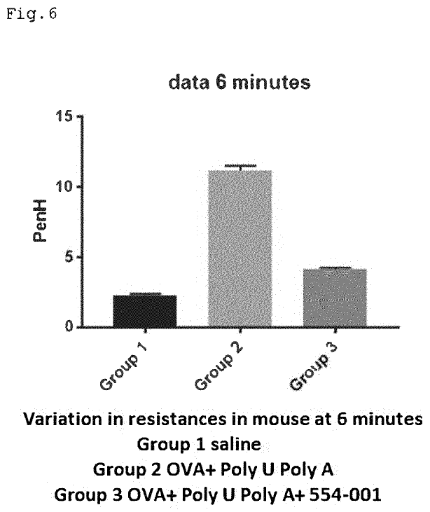

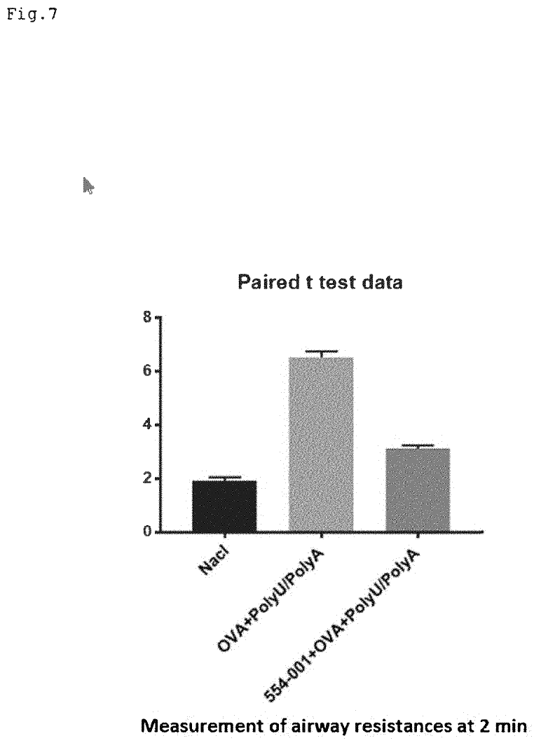

### DETAILED DESCRIPTION

To demonstrate that tritoqualine does inhibit respiratory distress syndrome via the basophil, a model described in publication of Ramadan 2013 has been used.

The inventors used a C57/B16J mouse model, hereafter referred to as BL6.

The protocol simulating a double-stranded RNA viral infection has been performed with 2 groups of 15 mice; these are 8 week old female mice. A control group treated only with saline served as a negative control.

BL6 mice are treated according to the protocol below. BL6 mice have been sensitised by intraperitoneal injections of 100 mg OVA (ovalbumin) on days 0, 2 and 4. Thereafter, from day 10 to day 15, the mice have received a daily treatment with aerosolized OVA at the concentration of 20 mg/ml or saline for 20 min, using an ultrasound nebulizer (Ultra-Neb99). One hour later, the mice received 50 mg of poly (A:U) or saline intranasally.

3 groups were studied:

Group 1 negative control which only receives saline.

Group 2 which is sensitised to OVA and also receives double-stranded RNA (Poly U/Poly A) according to the protocol described above.

Group 3 which is sensitised to OVA and also receives double stranded RNA (Poly U/Poly A) according to the protocol described above but also tritoqualine at 10 mg/kg.

Each group is analysed by whole body plethysmography and the measurements are expressed in PenH. A measurement is performed every minute until 10 minutes after the introduction of the double-stranded RNA. Tritoqualine treatment is given 1 hour before the introduction of Poly U/Poly A (double-stranded RNA).

In group 1, which is the negative control group there are the following results at time 1 minute to 10 minutes, expressed in PenH units and averaged over 15 mice:

TABLE 1

| | T1 | T2 | T3 | T4 | T5 | T6 | T7 | T8 | T9 | T10 |
|---|---|---|---|---|---|---|---|---|---|---|
| Group 1 | 1.83 | 1.91 | 2.04 | 2.08 | 2.27 | 2.30 | 2.25 | 2.15 | 2.15 | 2.08 |

For group 2, which is the positive control group (mice with viral infection after sensitisation to ovalbumin) there are have the following results at time 1 minute to 10 minutes (T1 to T10), expressed in PenH units and averaged over 15 mice:

TABLE 2

| | T1 | T2 | T3 | T4 | T5 | T6 | T7 | T8 | T9 | T10 |
|---|---|---|---|---|---|---|---|---|---|---|
| Group 2 | 5.1 | 6.5 | 7.2 | 8.2 | 9.5 | 11.2 | 10.6 | 9.6 | 8.9 | 8.1 |

In group 3, which is the group treated with tritoqualine at a dose of 10 mg/kg (mice with viral infection after sensitisation to ovalbumin as well) there are have the following results at time 1 minute to 10 minutes, expressed in PenH units and averaged over 15 mice:

TABLE 3

| | T1 | T2 | T3 | T4 | T5 | T6 | T7 | T8 | T9 | T10 |
|---|---|---|---|---|---|---|---|---|---|---|
| Group 3 | 3.05 | 3.11 | 3.13 | 3.97 | 4.07 | 4.14 | 4.02 | 3.85 | 3.14 | 2.74 |

If the different groups are compared, the group treated with tritoqualine shows an increase in its airway resistance, but only moderately compared to the untreated group. This difference is maximal at T 6 minutes between the tritoqualine group and the untreated group. The difference is still very significant at T 10 minutes.

Statistical analysis shows that the difference between these 2 groups is highly significant (p<0.001).

The conclusion is that in the model of respiratory inflammation by ovalbumin superinfected with the equivalent of a double-stranded RNA viral infection, tritoqualine shows surprising efficacy. It has been demonstrated in this model that it is the basophil that drives respiratory inflammation. (Ramadan 2013)

Tritoqualine is therefore capable of treating coronavirus infection-related respiratory distress.

The inventors have also tested tritoqualine directly on the basophil. For this purpose, the inventors have used a commercial test, called Flow Cast from Bülhmann.

This test uses the CD63 marker. The CD63 marker is considered as a marker of basophil and mast cell activation. Resting basophils express very little of the CD63 antigen because it is bound to intracytoplasmic granules.

Activation of basophils and mast cells leads to fusion of the granules with the plasma membrane and thus to CD63 expression on the cell surface.

CD63 therefore appears on the surface of basophils or mast cells only when CD63 is activated.

Activation of the basophil or mast cell can either be by an allergen or by an anti-IgE (anti-FcεRI, which is the anti-IgE receptor).

Activation of the basophil means that the basophil degranulates and releases many cytokines that are toxic to the lung. Indeed, it is in the lung that the toxic effect of the basophil is greatest. It is therefore imperative to block basophil degranulation to avoid toxic shock. In the scope of coronavirus infections, this toxic shock can lead to the patient's death.

Human mast cells and basophils are relatively similar in morphology, deriving from a CD34+ haematopoietic stem cell.

They differentiate under the influence of different cytokines, the main one being Stem Cell Factor for mast cells and Interleukin-3 for basophils. While mast cells are tissue-resident elements, the basophil is a circulating cell. It should be noted that mast cells represent a heterogeneous population depending on their tissue location, which is not the case for the basophil.

Both cells are involved in the IgE-dependent allergic reaction because they express the high-affinity IgE receptor. However, mediators released by these cells during this activation are in some cases different. In addition, basophils and especially mast cells are involved in innate immunity. It is in the context of innate immunity that the basophil is involved in coronavirus infection. Indeed, cytokine storm appears within 6 to 10 days after the start of the coronavirus infection. This is too short a time for acquired immunity to take place.

Basophils and mast cells are involved in many diseases through the secretion of many interleukins.

Mast cells and basophils secrete common cytokines such as IL 2, IL 3, IL 4, IL 5, IL 6, IL 9, IL 13, IL 15. Some of these interleukins are involved in allergy such as IL-4, others in pulmonary fibrosis such as IL-13, others in cytokine storm such as IL6. Basophils are also responsible for inflammatory reactions when activated. They degranulate to release histamine, proteoglycans such as heparin and chondroitin, and proteases such as elastase and lysophospholipase. They also secrete lipid mediators such as leukotrienes and various cytokines.

The inventors have highlighted the surprising role of tritoqualine in modulating CD63 and thus basophil degranulation. The inventors have highlighted the astonishing and surprising properties of tritoqualine in a human cell model of CD63 modulation.

To study this modulating action of CD63 by tritoqualine, the inventors have used the basophil and a modified commercial test, the Flow CAST® kit from BULHMANN Laboratories AG (Switzerland), which is a Basophil Activation Test (BAT) that can be used for the in vitro detection of basophil degranulation as well as for the study of immediate type allergic reactions and hypersensitivities.

The test is designed for the in vitro diagnosis of CD63 expression as a surface marker of activated basophils. The test is performed on whole blood; flow cytometry is used to quantify CD63 expression on the surface of activated basophils.

Activation (or degranulation) of basophils by this test can be made in three different ways:
by an allergen, or
by an “anti-IgE” (anti-FcεRI, which is the anti-IgE receptor) or
by a bacterial lipopolysaccharide antigen, called fMLP.

It is intended for in vitro diagnosis of CD63 expression, all on whole blood by flow cytometry after allergen stimulation.

Resting basophils express very little CD63 antigen because it is bound to intracytoplasmic granules.

Activation of basophils (for example by IgE (immunological activation) or fMLP (non-immunological activation)) leads to fusion of the granules with the plasma membrane and thus to CD63 expression on the cell surface.

To evaluate degranulation via CD63 expression, the Flow CAST® kit has been partially used. This test includes an anti-IgE receptor. The inventors have only used the latter activator.

On the other hand, the Flow Cast test uses a second membrane marker, CCR3. The protein encoded by this gene is a C—C type chemokine receptor. It belongs to the G-protein coupled receptor family 1.

It is highly expressed in eosinophils, basophils and is also detected in TH1 and TH2 cells and airway epithelial cells. This receptor may contribute to the accumulation and activation of eosinophils, basophils and other inflammatory cells in the allergic airways. This receptor coupled to cell size allows specific characterisation of the basophil. Analysis of these two receptors allows the basophil and its degranulation to be characterised in a highly specific manner by flow cytometry.

Flow cytometry is used to characterise the different blood cells.

Laser beams allow the evaluation and measurement of different cellular parameters:
The frontal measurement of the diffracted light of the laser beam allows evaluation of the cell size: this is the Forward SCatter (FSC).
The measurement of the perpendicularly diffracted light allows evaluation of the cell granularity: it is the Side SCatter (SSC).

This granularity may be due to irregularities internally to or at the surface of the cells or to the density of the organelles that compose it.

Fluorescence markers are then used to better characterise the different cell subpopulations (these markers are coupled with differentiation clusters).

The apparatus that has been used throughout these experiments is a BD FACS Canto flow cytometer with 2 lasers:

A blue one with the possibility of 3 frequencies: 564-606 nm, 515-545 nm, 750-810 nm.

A red one with the possibility of 2 frequencies: 750-810 nm and 650-670 nm.

Several fluorochromes are used: APC Cy 7 (APC-Cy™ 7 is a fluorochrome that combines APC and a cyanine dye) and FITC (fluorescein isothiocyanate) as well as PE (phycoerythrin).

These different couplings make it possible to characterise cells separately and in particular basophils.

These different couplings make it possible to characterise cells separately and in particular basophils. 4 windowing zones will be distinguished according to whether or not CD63 labelling is observed and whether or not CCR3 labelling is observed
CD63+ and CCR3− which characterise non-basophilic degranulated cells.
CD63+ and CCR3+ which characterise degranulated basophils.
CD63− and CCR3− which characterise non-degranulated non-basophilic cells.
CD63− and CCR3+ which characterise non-degranulated basophils.

Thus with this analysis it is possible to analyse degranulated basophils separately from non-degranulated basophils. It is thus possible, by interacting a molecule of therapeutic interest, to know its capacity or not to act on degranulation.

The degranulation protocol has been carried out using the Flow CAST.

The Flow CAST Kit is comprised of
A neutral stimulation Buffer with IL-3.
A Stimulation Buffer with anti-FcεRI antibody positive control (Stimulation Control).
A Stimulation Buffer with fMLP positive control (fMLP).

Not used in this protocol.
A Staining Reagent.
A Wash Buffer.
A Lysing Reagent.

Reminder: During basophil activation, CD63 markers bound to intra-cytoplasmic granules will fuse with the plasma membrane. They are then expressed on the cell surface: activated basophils hence become CD63+. Further to CD63, another basophil specific marker is CCR3 (chemokine receptor 3).
Activated and degranulated basophils are CD63+ and CCR3+;
non-degranulated basophils are CD63− and CCR3+.

Three tests have been made to define and refine the final protocol. Firstly, a “negative control” sample, that is containing only the neutral buffer, has been analysed in order to observe the results expected in the absence of stimulation and therefore of degranulation.

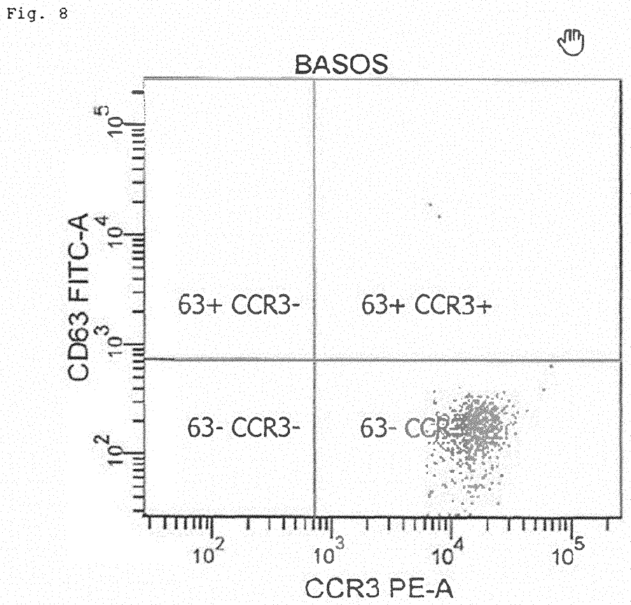

It can be noticed in [FIG. 8], which has 4 zones, that only the CD63− CCR3+ zone contains a scatter chart corresponding to non-degranulated basophils. In the absence of stimulation, the cells are not activated and do not degranulate.

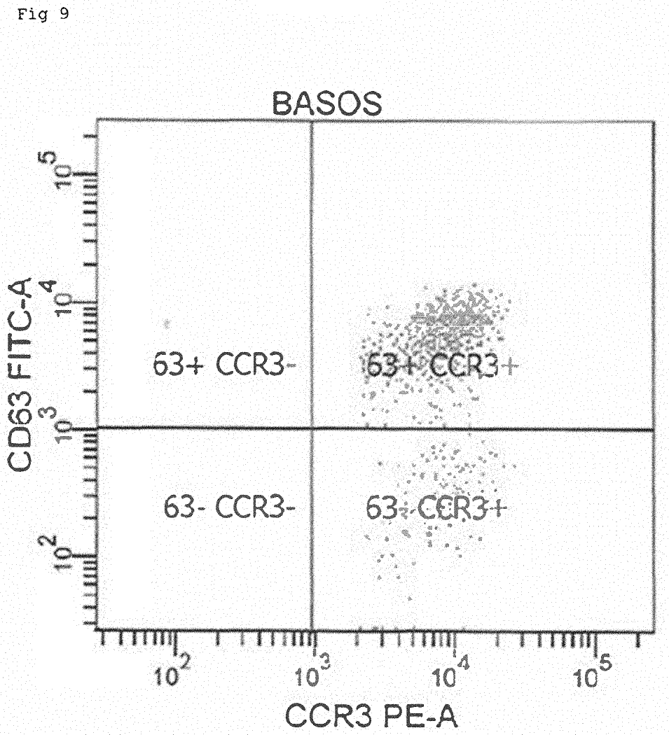

The second test comprises a “positive control” sample with the FcεRI antibody. On the 4 windowed zones in flow cytometry, a positive control Sample with FcεRI antibody window allows the observation of the scatter chart in the CD63+ CCR3+ zone. This is the zone in the upper right-hand corner of [FIG. 9]. This indicates that the anti-FcεRI antibody does cause degranulation of basophils. However, not all basophils are degranulated, as the CCR3+ and CD63− window contains a few spots corresponding to non-degranulated basophils.

Finally, the third test involves the use of the anti-FcεRI antibody for the purpose of degranulating the basophil with a prior incubation of tritoqualine at different concentrations. It is with this last analysis that the inventors were able to demonstrate activity of tritoqualine on CD63 modulation.

Finally, basophils are a very small population in the blood count, often less than 1%. With a goal of obtaining at least 500 basophils for analysis, more than 50,000 white blood cells had to be sorted on each passage in front of the flow cytometer.

Next, tritoqualine has been added to each sample with the anti-FcεRI antibody. If tritoqualine is able to inhibit degranulation, then the number of non-degranulated basophils should increase.

Concentrations of tritoqualine used range from 1 μM to 10 μM corresponding to therapeutic doses of 100 mg to 1 gram per day in a man having an average weight of 70 kg.

The experiments were as follows:

First, a patient has been sampled and 5 tubes have been prepared. One negative control tube: without incubation with tritoqualine and without the anti-FcεRI antibody (the degranulation product). Four “positive control” tubes: without incubation with tritoqualine and with the basophil degranulant (anti-FcεRI antibody).

The aim of these experiments was to demonstrate relevance of the test to differentiate degranulated from non-degranulated basophils.

The experiment confirmed that the anti-FcεRI antibody caused basophils to degranulate as in the series with the anti-FcεRI antibody (positive control), almost 85% of basophils degranulated. In the “negative control” sample, there are almost no degranulated cells (less than 17%).

Statistical analysis has been performed using GraphPad Prism 7.0.

The Student's t test of the negative control versus positive control also shows that p is less than 0.0001. This means that degranulated and non-degranulated basophils are highly differentiable in flow cytometry.

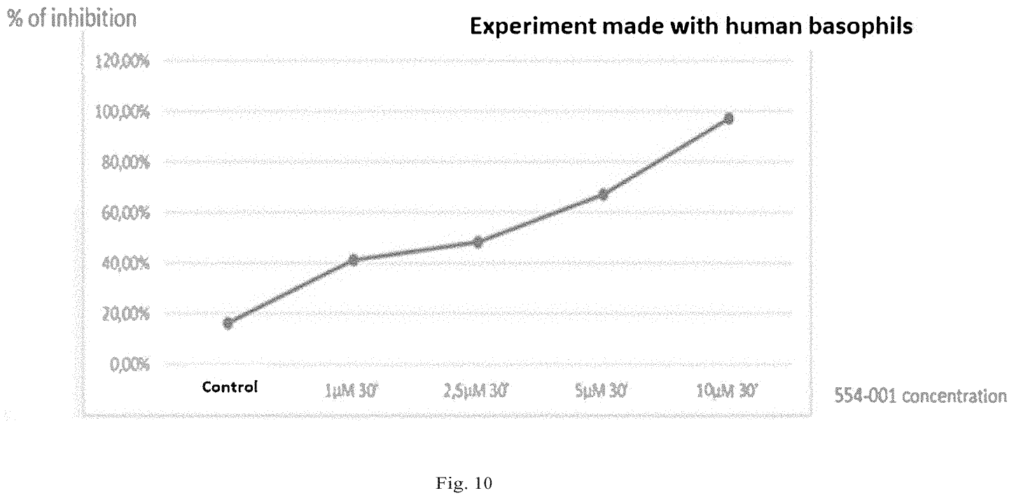

Thus, the study phase has been performed with 4 patients at doses ranging from 2.5 μmol to 10 μmol. By way of example, [FIG. 10] shows the dose effect of tritoqualine from 1 μmol to 10 μmol on the inhibition of the basophil degranulation and thus of the CD63 expression modulation.

At 5 μmol, CD63 is still expressed on approximately 40% of the cells whereas at 10 μmol, this expression is reduced to less than 2%. Statistical analysis shows a P greater than 0.001 for doses from 2.5 micromol to 10 micromol. Tritoqualine does modulate CD63 expression on basophils in a surprisingly progressive manner. This CD63 modulation makes it possible to contemplate tritoqualine-based therapies for the treatment of respiratory distress syndrome and coronavirus infection-related cytokine storm. This powerful degranulation inhibition action also makes it possible to contemplate the prevention of cytokine storm as well as coronavirus infection-related respiratory distress.

The treatment of these pathologies can be done at therapeutic doses between 5 mg/day and 700 mg/day.

Tritoqualine can be used in different forms, apart from its “compressed” form without modifying its efficacy, for example in the form of soft gelatin capsule, syrup or gel. 7-Amino-4,5,6-triethoxy-3-(5,6,7,8-tetrahydro-4-methoxy-6-methyl-1,3-dioxolo[4,5-g]isoquinolin-5-yl)phthalide can also be used in combination with complementary drugs, such as Nintedanib and Pirfenidone. These drugs acting with different modes of action, allow for lower doses and thus less side effects and toxicity. Nintedanib can be used at a dose of 10 mg to 50 mg/day.

Pirfenidone can also be used at lower doses such as 100 to 200 mg/day.

Commercial tritoqualine is a white powder, very sensitive to light which degrades it to Cotarnine and phthalic acid.

Tritoqualine has two asymmetric carbons, but analysis of the old commercial form shows that tritoqualine is a racemic mixture of 2 enantiomers (R—R and S—S) and not a mixture of 4 diastereomers.

Tritoqualine is a benzylisoquinoline with a molecular weight of 500. This compound can be modified or substituted with compounds comprising either carbon-14 or deuterated compounds.

Isotope-labelled compounds and salts can be used in a variety of ways. They may be suitable for drugs and/or different types of tests, such as tissue distribution tests on a substrate. For example, tritium and/or carbon-14 labelled compounds are particularly useful for various types of tests, such as tissue distribution tests on a substrate, due to their relatively simple preparation and excellent detectability. For example, deuterium labelled products are therapeutically useful and have potential therapeutic advantages over non-deuterium labelled compounds. In general, deuterium-labelled compounds and salts can have higher metabolic stability than non-deuterium-labelled compounds due to the isotope kinetic effect. Higher metabolic stability translates directly into an increased half-life in vivo or lower doses, which may be desired. Isotopically labelled compounds and salts can generally be prepared by following the procedures described in known synthesis schemes such as for example in patent EP3352757 filed by concert pharmaceuticals. It is thus easy to replace non-deuterated methyls with deuterated methyls. Thus 5 substitutions of deuterated methyls could be made on tritoqualine. Two on the cotarnine ring and 3 on the nitrophthalide ring.

Thus, it is possible to use these deuterated compounds to improve the bioavailability of tritoqualine and its efficacy in cytokine storm and the treatment of coronavirus infection-related respiratory distress.

1-5. (canceled)

6. A method of treating coronavirus infection-related cytokine storm in a subject, comprising administering to a subject in need thereof a therapeutically effective amount of 7-Amino-4,5,6-triethoxy-3-(5,6,7,8-tetrahydro-4-methoxy-6-methyl-1,3-dioxolo[4,5-g]isoquinolin-5-yl)phthalide and deuterated derivatives thereof.

7. A method of preventing coronavirus infection-related cytokine storm in a subject, comprising administering to subject in need thereof a therapeutically effective amount of 7-Amino-4,5,6-triethoxy-3-(5,6,7,8-tetrahydro-4-methoxy-6-methyl-1,3-dioxolo[4,5-g]isoquinolin-5-yl)phthalide and deuterated derivatives thereof.

8. A method of treating a coronavirus infection-related respiratory distress in a subject, comprising administering to a subject in need thereof a therapeutically effective amount of 7-Amino-4,5,6-triethoxy-3-(5,6,7,8-tetrahydro-4-methoxy-6-methyl-1,3-dioxolo[4,5-g]isoquinolin-5-yl)phthalide and deuterated derivatives thereof.

9. A method for preventing coronavirus infection-related respiratory distress in a subject, comprising administering to a subject in need thereof a therapeutically effective amount of 7-Amino-4,5,6-triethoxy-3-(5,6,7,8-tetrahydro-4-methoxy-6-methyl-1,3-dioxolo[4,5-g]isoquinolin-5-yl)phthalide and deuterated derivatives thereof.

10. The method according to claim 6, wherein the 7-Amino-4,5,6-triethoxy-3-(5,6,7,8-tetrahydro-4-methoxy-6-methyl-1,3-dioxolo[4,5-g]isoquinolin-5-yl)phthalide and deuterated derivatives thereof are packaged in the form of soft gelatin capsules, tablets, capsules, syrup or gel.
